(19) United States
(12) Patent Application Publication
(10) Pub. No.: US 2017/0014584 A1
(43) Pub. Date: Jan. 19, 2017

(54) INHALER MOUTHPIECE

(71) Applicant: Prakash MEHTA, Mumbai (IN)

(72) Inventor: Prakash MEHTA, Mumbai (IN)

(21) Appl. No.: 15/151,028

(22) Filed: May 10, 2016

(30) Foreign Application Priority Data
Jul. 15, 2015 (IN) 2676/MUM/2015

(51) Int. Cl. A61M 15/00

(52) U.S. Cl. CPC A61M 15/0025; A61M 15/009; A61M 15/0086; A61M 15/0023

(57) ABSTRACT

An improved metered dose inhaler may be provided. The improved inhaler may have a mouthpiece designed for improved medicine delivery. The mouthpiece may extend from a cap of the inhaler, which may be removable or molded to the body of the inhaler.

### PRIORITY

This application claims priority under 35 U.S.C. §119 to Indian Patent Application No. 2676/MUM/2015 filed Jul. 15, 2015, titled An Improved Metered Dose Inhaler And Spacers For Such Inhalers, the disclosure of which is incorporated by reference herein in its entirety.

### FIELD OF THE INVENTION

This invention relates to an inhaler, particularly to an improved metered dose inhaler and spacers for such Inhalers.

### BACKGROUND

I (the applicant) had been a smoker for many years, due to which I suffer from COPD (chronic obstructive pulmonary disease).

Around the world a large number of people suffer from various respiratory diseases, such as Asthma, Chronic Bronchitis, Emphysema etc.

A majority of them depend on MDI inhalers for treatment and relief. This group ranges from the very young, such as children, through all age groups, to the very senior people aged 80 years and above.

For all these people, in all age groups, afflicted with the whole range and spectrum of lung disease and damage, inhalers are a part of life and are life savers. But for most, and especially for children and the elderly, it is extremely difficult to use these inhalers in an easy and efficient manner.

In the traditional inhalers, you remove the cap, shake the inhaler and place the opening in the mouth, press to release the metered dose, and inhale.

The problems and difficulties with this type are as follows:

a) very inconvenient to place this opening comfortably in the mouth, especially for children and elders:
b) difficult to co-ordinate release and inhaling in, at the same time. (Spacers are available for this, where also our new device can be used. (Further notes below)
c) the powdered dose is not uniquely directed, so it spreads out in the mouth and less dosage enters the respiratory stream.

### SUMMARY OF THE INVENTION

In this invention the mouthpiece is a fixed part, either to the cap or the body of the MDI inhaler.

The mouthpiece of this device is a circular, (normally of diameter 5 mm to 10 mm, it can be of any other convenient shape, such as elliptical, oblong, rectangular, square etc.), hollow protrusion from the cap of the inhaler, of the required length (normally of 20 mm to 30 tnm). This mouthpiece is fixed and part of the cap. The cap, with this mouthpiece, can he removable or moulded as part of the body of the inhaler. It is therefore a part of the cap and for those who need to use this, just have to shake the inhaler, and use directly, with no need to remove the cap.

The same type of cap, of the required size to fit the outlet of the spacers, with the unique protruding/extended mouthpiece, to be used by fitting it to the inhaling end of such spacers.

“The cap, with this mouthpiece, can be removable or moulded as part of the body of the inhaler.”

The cap can be kept or removed as is warranted. All that is meant by this line is it can be a part of the body of the inhaler or a separate piece as is now available. (without the mouthpiece.).

This can be made of various materials.

### BRIEF DESCRIPTION OF THE DRAWINGS

FIG. 1 shows the prospective view of the inhaler.

FIG. 2 shows the prospective view of the inhaler from a different angle.

FIG. 3 shows the internal view of the mouth piece.

FIG. 4 shows the prospective view of the mouth piece with protrusion.

FIG. 5 shows the side view of the mouth piece with protrusion.

FIG. 6 shows the prospective view of the mouth piece (angular view).

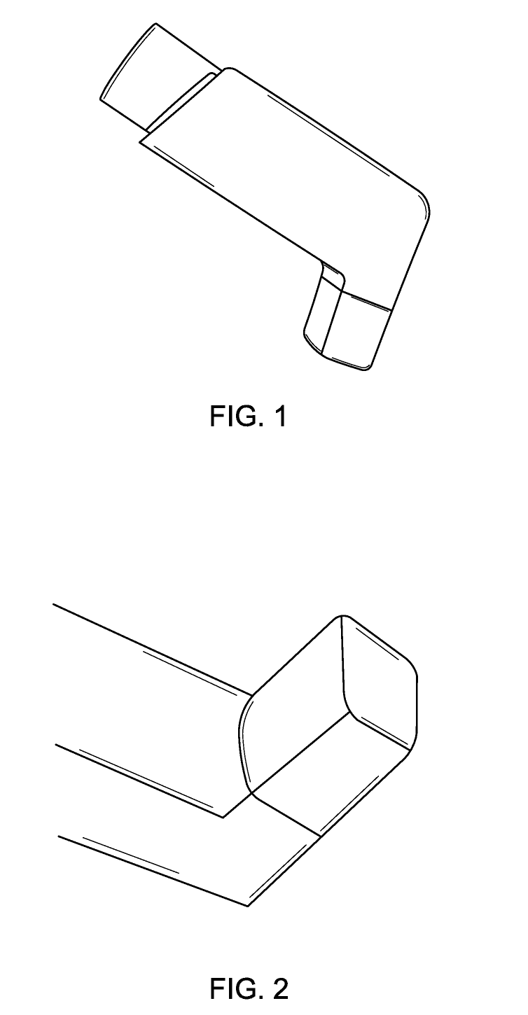

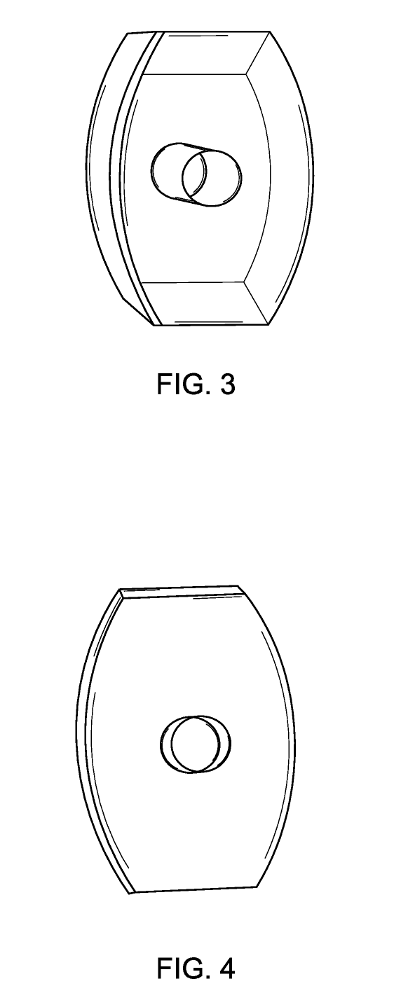

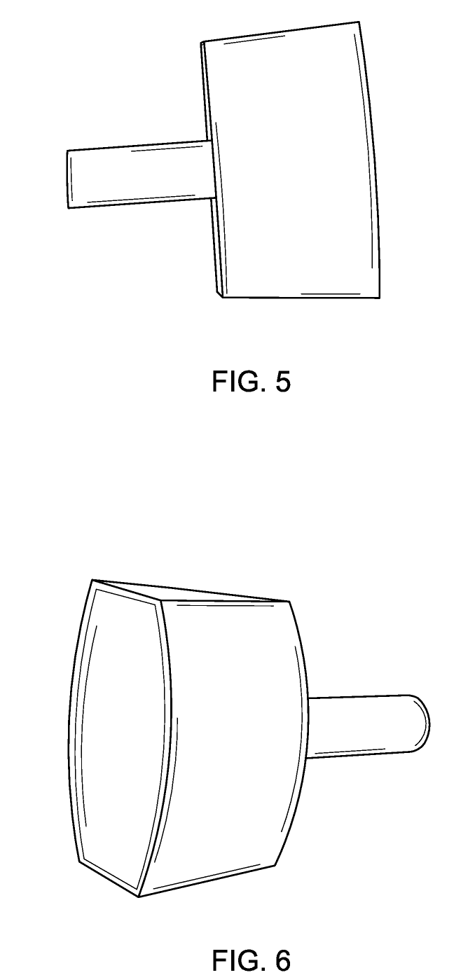

### DETAILED DESCRIPTION

The main object of the invention is to obviate the drawbacks in the prior art.

The instant invention provides the following benefits:

Referring to the Figures generally, the new design/device improves the inhaler and its efficiency in the following manner:

Nearly everyone is familiar, and most likely use a straw to suck in and drink various forms of liquid, such as soft drinks, juices, shakes etc. And do so very easily, efficiently and without strain whatsoever.

This devices mouthpiece is easy and comfortable to place in the mouth. This circular protruding part can be of any shape (elliptical, square, oblong, rectangle etc) and length (also pulling in and out to adjust the required length), it can be part of the cap, or it can be part of the casing of the compressed capsule/cylinder of the inhaler. It can be shaped into various forms, by itself or at its attachment with the cap, or casing, and can be made of various materials.

As it is a familiar mouth apparatus, like the daily use straw, it is very, very simple and easy to co-ordinate the breathing in (inhaling) while pressing the inhaler to release the dose. The user has to just suck in the dose. This is especially useful and helpful for young children and elderly people. The same device (cap with a protruding hollow tube/apparatus) can be fitted to the mouth piece of various types of spacers, for the same convenience, effectiveness and efficiency.

This device sends out a very precise stream of the medication that is directed efficiently to the back of the mouth/throat so that it easily and rapidly enters the air flow into the lungs, with more of the medication. This makes it a unique device for the satisfaction of the user, as it is easy to use, simple and convenient. There is less or no strain, more precise and efficient, gives better results and is absolutely the most comfortable and practical method for all, especially small children and old people, more so with those with weaker lungs.

It has been designed so that it is comfortable for person to use wherein the dimension and size is such that pushes a precise flow directly in ifs normal breathing flow into the lungs (direct medication without loss of medicine) since the cylinder is well fitted into the mouth there is no leakage of the medication from the lips where as it happens when you use the regular mouth of the inhaler (the big piece)

it is easy to use, precise flow, matches the flow of breathing air into the lungs. The mouth piece is properly sealed by the lips so that no leakage of that flow.

While the bigger piece is uncomfortable, pressing the cylinder is difficult, when medication is taken user opens his mouth and some medication is lost.

A greater portion of the metered dose of medication is inhaled by use of this new device compared to the order open mouth.

When the medication which is coming out the mouth when using the regular mouth piece is that there is a back flow.

According to an exemplary embodiment, an improved metered dose inhaler may be provided. The inhaler may have a mouthpiece which is a fixed part attached either to the cap or the body of the MDI Inhaler. The mouthpiece may be a convenient shape, hollow protrusion from the cap of the inhaler, of the required length (normally of 20 mm to 30 mm). The mouthpiece may be fixed and part of the cap. The cap, with this mouthpiece, can be removable or moulded as part of the body of the inhaler. The same type of cap, of the required size to fit the outlet of the spacers, with the unique protruding/extended mouthpiece, to be used by fitting it to the inhaling end of such spacers. The mouthpiece may be of a convenient shape such as circular, elliptical, oblong, rectangular, square etc. The diameter of the mouth piece may have a diameter of approximately 5 mm to approximately 10 mm. The cap of the improved metered dose inhaler with the mouthpiece may be removable or molded as part of the body of the inhaler. The mouthpiece may have been designed so that it is comfortable for person to use wherein the dimension and size is such that it pushes a precise flow directly in a normal breathing flow into the lungs (direct medication without loss of medicine) since the cylinder is well fitted into the mouth there is no leakage of the medication from the lips where as it happens when you use the regular mouth of the inhaler (the big piece). It is easy to use, has precise flow, and matches the flow of breathing air into the lungs. The mouthpiece may be properly sealed by the lips so that no leakage or “back flow” occurs, as happens with regular inhalers. The various parts of the inhaler may be made of various materials.

The present invention is not limited to the above-described embodiments, and various alterations, modifications, and/or alternative applications of the invention may be possible, if desired, without departing from the scope and sprit of the invention, which can be read from the entire specification. All these possible alterations, modifications, and/or alternative applications of the invention are also intended to be within technical scope of the present invention.

I claim:

1. An improved metered dose inhaler comprising:
an inhaler body;
a cap; and
a mouthpiece attached to the cap, wherein the mouthpiece is a hollow protrusion extending from the cap, and wherein the cap is removable from the inhaler body or integrally molded as part of the inhaler body.

2. The inhaler of claim 1, wherein the mouthpiece is between 20 mm and 30 mm long.

3. The inhaler of claim 1, wherein the cap is configured to fit the inhaling outlet of spacers.

4. The inhaler of claim 1, wherein the mouthpiece has one of a circular, elliptical, oblong, rectangular, or square cross-section.

5. The inhaler of claim 1, wherein the diameter of the mouthpiece is between 5 mm and 10 mm.

6. The inhaler of claim 1, wherein the mouthpiece is configured to fit within a user's mouth and deliver a precise flow of medicine in the user's normal breathing flow without leaking from the user's lips.

7. The inhaler of claim 1, wherein the mouthpiece is slidable in relation to the cap, such that the mouthpiece can retract or extend further from the cap.
